(19) United States
(12) Patent Application Publication
(10) Pub. No.: US 2024/0123039 A1
(43) Pub. Date: Apr. 18, 2024

(54) DETOXIFICATION PREPARATIONS WITH REINFORCED BOOSTERS TO TREAT ALCOHOL INTOXICATION

(71) Applicant: Richard C.K. Yen, Yorba Linda, CA (US)

(72) Inventor: Richard C.K. Yen, Yorba Linda, CA (US)

(21) Appl. No.: 18/485,561

(22) Filed: Oct. 12, 2023

Related U.S. Application Data
(60) Provisional application No. 63/475,146, filed on Oct. 18, 2022.

(51) Int. Cl. A61K 38/44; A61K 9/48; A61K 47/26; A61P 25/32

(52) U.S. Cl. CPC A61K 38/44; A61K 9/4816; A61K 38/443; A61K 47/26; A61P 25/32; C12Y 101/01001; C12Y 102/01003

(57) ABSTRACT

A composition and method for converting alcohol to acetaldehyde and/or acetate and treat alcohol intoxication. The composition can include a therapeutically effective amount of an enzyme and a therapeutically effective amount of a co-factor. The enzymes can be alcohol dehydrogenase (ADH), a derivative of alcohol dehydrogenase, an analog of alcohol dehydrogenase or alcohol dehydrogenase in yeast crude extract. A second enzyme can be aldehyde dehydrogenase (ALDH), a derivative of aldehyde dehydrogenase, an analog of aldehyde dehydrogenase, mitochondrial aldehyde dehydrogenase (ALDH2), a derivative of mitochondrial aldehyde dehydrogenase, an analog of mitochondrial aldehyde dehydrogenase or aldehyde dehydrogenase in yeast crude extract. A co-factor can be utilized in the composition that increases a concentration of Nicotinamide Adenine Dinucleotide (NAD) or Nicotinamide Adenine Dinucleotide Phosphate (NADP). A sugar such as, but not limited to, fructose and a third enzyme sorbitol dehydrogenase (SDH) can be included in the composition.

### CROSS-REFERENCE TO RELATED APPLICATION

This application claims the benefit of priority under 35 U.S.C. § 119(e) based upon co-pending U.S. provisional patent application Ser. No. 63/475,146 filed on Oct. 18, 2022 entitled “Detoxification Preparations to Remove Harmful Chemicals from Alcohol Consumption”. The entire disclosure of the prior provisional application is incorporated herein by reference.

### BACKGROUND

### Technical Field

In some aspects, the present technology relates to detoxification preparations with reinforced boosters to treat alcohol intoxication. In some other aspects, the present technology relates to preparations in liquid, semi-liquid forms or solid/gel capsules with a reinforced booster cofactor system that can detoxify the effects of alcohol consumption. In still some other aspects, the present technology relates to methods of using a preparation or composition that detoxifies the effects of alcohol consumption.

### BACKGROUND DESCRIPTION

Consumption of excessive alcoholic drinks can lead to, but not limited to, dysphoria, social embarrassment and illnesses including alcoholism, and alcohol-related cancer.

Alcohol treatment centers are centers set up specifically for the care of people dealing with alcoholism. One example of such a center called Sabino Recovery stated the following: “We recognize that addiction is multifaceted and there is not a one-size-fits-all method for recovery. We are only able to effectively treat the issue by understanding the root cause. Many people who come to Sabino Recovery for help with addiction may not identify past trauma as one of the reasons they are struggling with addiction, so this program is specifically designed to treat addiction and the underlying trauma that may be fueling it.” This statement admits that there is not one particularly effective method of removing the harm of alcohol addiction, or even short term excessive alcoholic consumption.

Medications typically used to treat alcoholism include.
1. Naltrexone
2. Disufiram/Antabuse
3. Acamprosate
4. Gabapentin
5. Baclofen
6. Topiramate and others.

The mechanism of Naltrexone on the reduction of alcohol use is similar to that of reducing opiate use, by blocking the endorphin rush associated with either substance. It is not specific for the reduction of the toxic chemicals formed during the metabolism of ethanol in the body, specifically the highly toxic effect of acetaldehyde, the immediate break-down product of alcohol in the body.

Disulfiram/Antabuse works by blocking the enzyme responsible for the breakdown of acetaldehyde, which will build up in the body of the person taking this medication, leading to unpleasant sensations, which is supposed to be a strong dis-incentive so that the person will stop taking alcoholic drinks. It builds up the toxic substance (acetaldehyde) in the body instead of reducing or eliminating it.

Acamprosate works by promoting a balance between the excitatory and inhibitory neurotransmitters, which are: glutamate and gamma-aminobutyric acid, respectively. It may also reduce withdrawal-associated stress. Again, this drug does not remove the toxic chemicals resulting from alcohol consumption.

Gabapentin is not approved by the FDA for alcohol treatment. Gabapentin is originally developed to treat epilepsy and is now deemed helpful in people with anxiety or insomnia.

Baclofen is a muscle relaxant typically used to treat muscle spasms in people with multiple sclerosis or spinal cord defects. It appears to reduce binge drinking and helps in controlling cravings for alcohol. It does not eliminate the harmful substances formed from alcoholic consumption.

Topiramate is typically used to treat seizures and migraines. However, people taking it appear to have fewer cravings and have less pleasure from alcohol consumption.

### SUMMARY

In view of the foregoing disadvantages inherent in the known types of alcoholism treatment medications at least some embodiments of the present technology provides a novel dismountable detoxification preparations with reinforced boosters to treat alcohol intoxication, and overcomes one or more of the mentioned disadvantages and drawbacks of the prior art. As such, the general purpose of at least some embodiments of the present technology, which will be described subsequently in greater detail, is to provide a new and novel detoxification preparations with reinforced boosters to treat alcohol intoxication which has all the advantages of the prior art mentioned herein and many novel features that result in a detoxification preparations with reinforced boosters to treat alcohol intoxication which is not anticipated, rendered obvious, suggested, or even implied by the prior art, either alone or in any combination thereof.

According to one aspect, the present technology can include a composition for converting alcohol to acetaldehyde and/or acetate. The composition can include a therapeutically effective amount of one or more first enzymes, and a therapeutically effective amount of one or more second enzymes different to that of the first enzymes.

According to another aspect, the present technology can include a composition for converting alcohol to acetaldehyde and/or acetate. The composition can include a therapeutically effective amount of one or more first enzymes, a therapeutically effective amount of one or more second enzymes different to that of the first enzymes, and a therapeutically effective amount of one or more co-factors.

According to yet another aspect, the present technology can include a composition for converting alcohol to acetaldehyde and/or acetate. The composition can include a therapeutically effective amount of one or more first enzymes, a therapeutically effective amount of one or more second enzymes different to that of the first enzymes, a therapeutically effective amount of one or more co-factors, and a therapeutically effective amount of one or more sugars. According to still yet another aspect, the present technology can include a method for detoxify an effect of alcohol consumption in a subject. The method can include consuming a composition by a subject before and/or after consuming ethanol. The composition can include a therapeutically effective amount of one or more first enzymes, and a therapeutically effective amount of one or more second enzymes different to that of the first enzymes.

According to yet still another aspect, the present technology can include a method for in vivo reducing acetaldehyde levels and/or increasing a rate of catabolism of acetaldehyde. The method can include contacting at least one cell with an effective amount of a composition that modulates an enzymatic activity of an acetaldehyde dehydrogenase found in the at least one cell. The composition can include a therapeutically effective amount of one or more first enzymes, and a therapeutically effective amount of one or more second enzymes different to that of the first enzymes.

In some embodiments, the first enzymes can be selected from the group consisting one of or any combination of alcohol dehydrogenase (ADH), a derivative of alcohol dehydrogenase, and an analog of alcohol dehydrogenase.

In some embodiments, the alcohol dehydrogenase can be alcohol dehydrogenase in yeast crude extract.

In some embodiments, the second enzymes can be selected from the group consisting one of or any combination of aldehyde dehydrogenase (ALDH), a derivative of aldehyde dehydrogenase, an analog of aldehyde dehydrogenase, mitochondrial aldehyde dehydrogenase (ALDH2), a derivative of mitochondrial aldehyde dehydrogenase, and an analog of mitochondrial aldehyde dehydrogenase.

In some embodiments, the aldehyde dehydrogenase or the mitochondrial aldehyde dehydrogenase can be aldehyde dehydrogenase in yeast crude extract.

Some embodiments of the present technology can include a therapeutically effective amount of a co-factor configured to increase a concentration of any one of or any combination of Nicotinamide Adenine Dinucleotide (NAD) or Nicotinamide Adenine Dinucleotide Phosphate (NADP) in vivo.

In some embodiments, the co-factor can be selected from the group consisting one of or any combination of Nicotinamide Riboside (NR), and Nicotinamide Mononucleotide (NMN). In some embodiments, a concentration of the co-factor in the composition can be configured to prevent conversion of acetaldehyde to alcohol or acetate to acetaldehyde.

Some embodiments of the present technology can include an effective amount of any one of or any combination of a sugar, and a third enzyme different to that of the first and second enzymes.

In some embodiments, the sugar can be fructose, and the third enzyme can be sorbitol dehydrogenase (SDH).

In some embodiments, the composition can be selected from the group consisting of a pharmaceutical composition, a dietary supplement, a nutraceutical, a medicament, a liquid composition, a confection, and a powder.

Some embodiments of the present technology can include a pharmaceutically acceptable excipient.

In some embodiments, an amount of the composition can be a prophylactically effective amount.

In some embodiments, a number of units of any one of or any combination of the first enzymes and the second enzymes can be based on a value associated with a curve generated by the first and second enzymes, respectively, in a crude extract with a value associated with a curve generated by a pure enzyme preparation.

In some embodiments, the number of units can be based on a millimole value of ethanol divided by a time value in minutes.

There has thus been outlined, rather broadly, features of the present technology in order that the detailed description thereof that follows may be better understood and in order that the present contribution to the art may be better appreciated.

Numerous objects, features and advantages of the present technology will be readily apparent to those of ordinary skill in the art upon a reading of the following detailed description of the present technology, but nonetheless illustrative, embodiments of the present technology when taken in conjunction with the accompanying drawings.

As such, those skilled in the art will appreciate that the conception, upon which this disclosure is based, may readily be utilized as a basis for the designing of other structures, methods and systems for carrying out the several purposes of the present technology. It is, therefore, that the claims be regarded as including such equivalent constructions insofar as they do not depart from the spirit and scope of the present technology.

It is therefore an object of the present technology to provide a new and novel detoxification preparations with reinforced boosters to treat alcohol intoxication that has all of the advantages of the prior art alcoholism treatment medications and none of the disadvantages.

It is another object of the present technology to provide a new and novel detoxification preparations with reinforced boosters to treat alcohol intoxication that may be easily and efficiently manufactured and marketed.

An even further object of the present technology is to provide a new and novel detoxification preparations with reinforced boosters to treat alcohol intoxication that has a low cost of manufacture with regard to both materials and labor, and which accordingly is then susceptible of low prices of sale to the consuming public, thereby making such detoxification preparations with reinforced boosters to treat alcohol intoxication economically available to the buying public.

Still another object of the present technology is to provide a new detoxification preparations with reinforced boosters to treat alcohol intoxication that provides in the apparatuses and methods of the prior art some of the advantages thereof, while simultaneously overcoming some of the disadvantages normally associated therewith.

These together with other objects of the present technology, along with the various features of novelty that characterize the present technology, are pointed out with particularity in the claims annexed to and forming a part of this disclosure. For a better understanding of the present technology, its operating advantages and the specific objects attained by its uses, reference should be made to the accompanying drawings and descriptive matter in which there are illustrated embodiments of the present technology. Whilst multiple objects of the present technology have been identified herein, it will be understood that the claimed present technology is not limited to meeting most or all of the objects identified and that some embodiments of the present technology may meet only one such object or none at all.

### BRIEF DESCRIPTION OF THE DRAWINGS

The present technology will be better understood and objects other than those set forth above will become apparent when consideration is given to the following detailed description thereof, with phantom lines (long-short-short-long lines) depicting environmental structure and forming no part of the claimed present technology. Such description makes reference to the annexed drawings wherein:

FIG. 1 is a schematic representational view of the metabolism of ethanol to acetaldehyde to acetate.

FIG. 2 is a schematic representational view of the important role of co-factors.

FIG. 3 is a schematic representational view of both ADH and ALDH need NAD as co-factor to oxidize alcohol to acetate.

FIG. 4 is a schematic representational view of the Reverse reaction using up NADH to replenish NAD.

FIG. 5 is a schematic representational view of the build-up of NAD from NR or NMN.

FIG. 6 is a graphical view of the OD of samples from Table 2 after subtracting OD at time zero vs. Time.

FIG. 7 is a graphical view of the OD of samples from Table 3 after subtracting OD at time zero vs. Time.

The same reference numerals refer to the same parts throughout the various figures.

### DETAILED DESCRIPTION OF THE EMBODIMENTS

Apart from the ineffectiveness and/or disadvantages of known alcoholism treatment medications. While the above-described devices fulfill their respective, particular objectives and requirements, the aforementioned devices or systems do not describe a detoxification preparations with reinforced boosters to treat alcohol intoxication. The present technology additionally overcomes one or more of the disadvantages associated with the prior art by that the present technology eliminates or greatly reduces the chemical entities responsible for the undesirable effects resulting from alcohol consumption.

Therefore, the prior art in the treatment of inappropriate alcohol consumption does not remove the toxic effect of either the alcohol itself or the toxic effect of its metabolites. There remains a need for a treatment that is easily administered at the time and the site of alcohol consumption, which eliminates or greatly reduces the chemical entities responsible for the undesirable effects resulting from alcohol consumption.

A need exists for a new and novel detoxification preparations with reinforced boosters that can be used to treat alcohol intoxication. In this regard, the present technology substantially fulfills this need. In this respect, the detoxification preparations with reinforced boosters to treat alcohol intoxication according to the present technology substantially departs from the conventional concepts and designs of the prior art, and in doing so provides an apparatus primarily developed for the purpose of to treat alcohol intoxication.

In the following description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

The present technology contemplates a preparation which is a solution of enzymes, co-enzymes (also called co-factors) and other substrates (or precursors of the substrates) which will have the effect of (a) quickly lowering the concentration of alcohol (ethanol or methanol); (b) lowering the concentration of a very toxic compound called acetaldehyde, (c) lowering quickly both the concentration of alcohol and acetaldehyde, so that the toxic and unacceptable effect of either or both chemicals can be lowered to a non-toxic and acceptable level in the body.

The present technology solution mentioned above can be taken orally. The taste of the solution/preparation would be acceptable to most people whether they have ingested alcohol or not. In many cultures it is considered rude to the host if the invited guest does not finish the alcoholic drink in a short time, even though the invited guest may not tolerate the effects of alcohol. In that case, the invited guest may orally intake the detoxification preparation of the present technology before, or at the same time he takes a sip of the alcoholic drink. For oral intake, the preparation may mainly detoxify the alcohol and its metabolites in the stomach and the intestine, but it is possible that in some cases, the alcohol in the blood may be so high compared to the level of alcohol in the digestive system that by “simple diffusion” (following the path of high concentration to the location with low concentration) it may “reverse” its path and re-enter the digestive system from the blood, to become affected by the preparation within the digestive system (mainly the stomach and the intestine, which have plenty of the enzymes to digest alcohol). Many of the components of this preparation can also enter into the blood system and work there (not being confined to the digestive system). In addition, the liver is the main organ for the removal of alcohol—and it is expected that the present technology preparation to have a positive effect on the effectiveness of the host's liver to detoxify the ingested alcohol (including the alcohol which has reached the liver via the circulatory system).

The present technology solution mentioned above can (as an option) also be administered intravenously in a situation where much of the alcohol or its metabolites have reached the blood stream and the person needs to be detoxified quickly. In this situation, the preparation must meet the requirements of an injectable preparation, i.e. being osmolarity compatible and without contaminants that may not be compatible with intravenous administration.

The present technology can also include the preparation of the same enzymes for oral intake but as a candy (hard or soft) or as a chewable such as, but not limited to, a gummy-bear type of chewable candy. The material used to make the candy may contain material that will protect the enzymes against digestion or destruction by the gastric contents or delay the destructive effects of digestive enzymes.

The present technology can also include the preparation of enzymes as a dry powder or solid, which can be packaged as a pouch inside a moisture-resistant pouch, to be ingested as solids or semi-solid pastes. This can be mixed with other food so that the composition is acceptable to a person who may not otherwise accept the product, possibly due to paranoia or other side-effects of intoxication from alcohol. Alternatively, the solid or dry-powder detoxification preparation can be dissolved in water or other suitable liquids to be taken as a drink.

The term “alcohol” herein includes the class of organic molecules commonly known as alcohol, which typically includes methanol, ethanol and propanol. Alcoholic drinks should contain only ethanol which can be metabolized by the body without major ill effects unless an excessive amount is consumed, or over a long period. Consumption of drinks containing methanol or propanol or other alcohols can lead to immediate medical problems. For the sake of discussion, this disclosure will only use examples from ethanol (or ethyl alcohol), but the treatments disclosed in the present technology can readily be applied to the patient who unfortunately or unintendedly consumed the other kinds of alcohol.

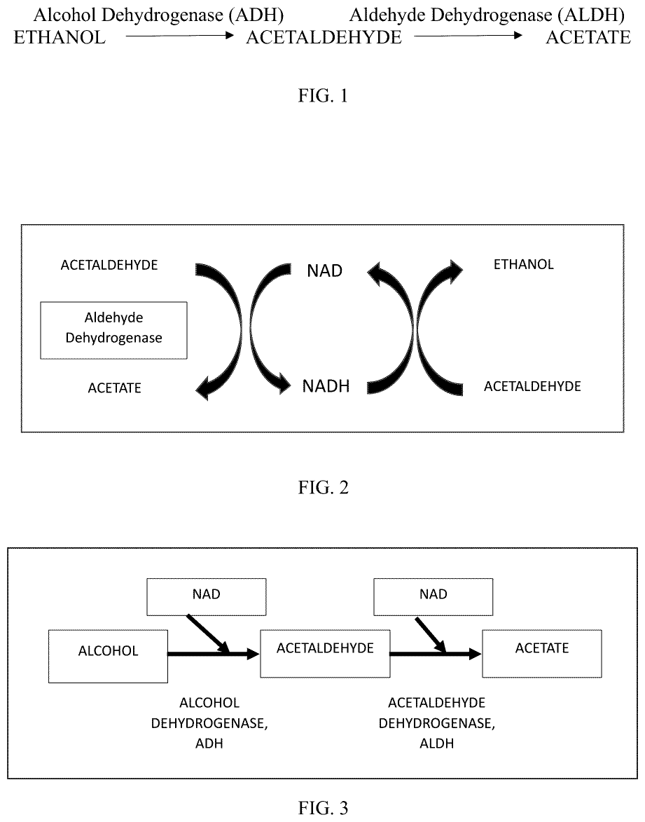

The metabolism of alcohol is well known. For details, one may read the “Alcohol Alert” from the National Institute of Health, National Institute on Alcohol Abuse and Alcoholism, Number 72, April 2007—“Alcohol Metabolism: An Update.” FIG. 1 describes the metabolism of ethanol to acetaldehyde to acetate, being the breakdown of ethanol into acetaldehyde by the enzyme called alcohol dehydrogenase (ADH). Acetaldehyde is sometimes called the “intermediate product”—it is highly toxic and is broken down into acetate by another or second enzyme called aldehyde dehydrogenase (ALDH).

There are other enzymes in the body which can also catalyze the conversion of ethanol into acetaldehyde, including P4502E1 in the microsome, any ADH Isozymes, cytochrome P450 (CYP2E1) and catalase in the peroxisomes. In this disclosure the present technology can include any and/or all enzymes which can convert ethanol into acetaldehyde when the present description uses the term ADH or alcohol dehydrogenase.

According to one aspect, the present technology can include a composition for converting alcohol to acetaldehyde and/or acetate. The composition can include a therapeutically effective amount of one or more first enzymes, and a therapeutically effective amount of one or more second enzymes different to that of the first enzymes.

According to another aspect, the present technology can include a composition for converting alcohol to acetaldehyde and/or acetate. The composition can include a therapeutically effective amount of one or more first enzymes, a therapeutically effective amount of one or more second enzymes different to that of the first enzymes, and a therapeutically effective amount of one or more co-factors.

According to yet another aspect, the present technology can include a composition for converting alcohol to acetaldehyde and/or acetate. The composition can include a therapeutically effective amount of one or more first enzymes, a therapeutically effective amount of one or more second enzymes different to that of the first enzymes, a therapeutically effective amount of one or more co-factors, and a therapeutically effective amount of one or more sugars.

According to still yet another aspect, the present technology can include a method for detoxify an effect of alcohol consumption in a subject. The method can include consuming a composition by a subject before and/or after consuming ethanol. The composition can include a therapeutically effective amount of one or more first enzymes, and a therapeutically effective amount of one or more second enzymes different to that of the first enzymes.

According to yet still another aspect, the present technology can include a method for in vivo reducing acetaldehyde levels and/or increasing a rate of catabolism of acetaldehyde. The method can include contacting at least one cell with an effective amount of a composition that modulates an enzymatic activity of an acetaldehyde dehydrogenase found in the at least one cell. The composition can include a therapeutically effective amount of one or more first enzymes, and a therapeutically effective amount of one or more second enzymes different to that of the first enzymes.

In some embodiments, the first enzymes can be selected from the group consisting one of or any combination of alcohol dehydrogenase (ADH), a derivative of alcohol dehydrogenase, and an analog of alcohol dehydrogenase.

In some embodiments, the alcohol dehydrogenase can be alcohol dehydrogenase in yeast crude extract.

In some embodiments, the second enzymes can be selected from the group consisting one of or any combination of aldehyde dehydrogenase (ALDH), a derivative of aldehyde dehydrogenase, an analog of aldehyde dehydrogenase, mitochondrial aldehyde dehydrogenase (ALDH2), a derivative of mitochondrial aldehyde dehydrogenase, and an analog of mitochondrial aldehyde dehydrogenase.

In some embodiments, the aldehyde dehydrogenase or the mitochondrial aldehyde dehydrogenase can be aldehyde dehydrogenase in yeast crude extract.

Some embodiments of the present technology can include a therapeutically effective amount of a co-factor configured to increase a concentration of any one of or any combination of Nicotinamide Adenine Dinucleotide (NAD) or Nicotinamide Adenine Dinucleotide Phosphate (NADP) in vivo.

In some embodiments, the co-factor can be selected from the group consisting one of or any combination of Nicotinamide Riboside (NR), and Nicotinamide Mononucleotide (NMN).

In some embodiments, a concentration of the co-factor in the composition can be configured to prevent conversion of acetaldehyde to alcohol or acetate to acetaldehyde.

Some embodiments of the present technology can include an effective amount of any one of or any combination of a sugar, and third enzyme different to that of the first and second enzymes.

In some embodiments, the sugar can be fructose, and the third enzyme is sorbitol dehydrogenase (SDH).

In some embodiments, the composition can be selected from the group consisting of a pharmaceutical composition, a dietary supplement, a nutraceutical, a medicament, a liquid composition, a confection, and a powder.

Some embodiments of the present technology can include a pharmaceutically acceptable excipient.

In some embodiments, an amount of the composition can be a prophylactically effective amount.

In some embodiments, a number of units of any one of or any combination of the first enzymes and the second enzymes can be based on a value associated with a curve generated by the first and second enzymes, respectively, in a crude extract with a value associated with a curve generated by a pure enzyme preparation.

In some embodiments, the number of units can be based on a millimole value of ethanol divided by a time value in minutes.

Regarding FIG. 1, it should be noted that the arrows point from ethanol toward acetaldehyde, then toward acetate (to be called “left to right” in this disclosure), because that is the usual direction of flow. However, in the real world of enzyme activity, enzymes are capable of conducting reversible reactions, i.e. acetaldehyde can be converted back to ethanol, if the concentration of acetaldehyde is high enough and the concentration of ethanol is low enough. In other words, the enzyme is not committed to only one direction of synthesis, but the direction of synthesis depends on the relative abundance (concentration) of (a) substrate versus (b) product. If for any reason, the “product” concentration is accumulating, the same enzyme may start reversing the reaction, and use the (b)-chemical as a “substrate”, to synthesize (a), which now becomes the “product.”

The other determinant which often dictates which chemical or compound serves as “substrate” versus which other compound becomes the “product” is the concentration of “co-factors” or “co-enzymes.” Co-factors are chemicals themselves, which is needed for a reaction to proceed, often as “hydrogen-acceptors” or “hydrogen-donors.” FIG. 2 illustrates the important role of co-factors and introduces the co-factors NAD (Nicotinamide Adenine Dinucleotide) and its reduced form NADH. These co-factors serve as electron carriers: the “oxidized” form being NAD and the corresponding “reduced” chemical is NADH.

It should be noted that in every (typical) enzyme reaction, if one substrate is oxidized, the other substrate is reduced. Historically, the removal of ethanol (or its lower concentration) is of great interest and therefore, the enzyme is called “alcohol” dehydrogenase (and not “NAD hydrogenase”). Actually, NAD is the other substrate and a very important one. Without the presence of NAD, the enzyme ADH will not be able to catalyze the reaction: this is because for one molecule of Ethanol to be oxidized (to Acetaldehyde), there must be a molecule of NAD to be reduced (to NADH). The reason why the same enzyme is not called NAD-hydrogenase is because (a) historically biochemists work on interesting compounds like ethanol, to see what the ethanol molecule would become if it is broken down, and (b) NAD appears to be all over the place (and not restricted to only one reaction). Therefore, the NAD was regarded only as a “co-factor” and not the “main character.” However, the co-factor is exceedingly important—without the presence of an adequate concentration of NAD (or its equivalent Nicotinamide Adenine Dinucleotide Phosphate (NADP)) the reaction will lack a “hydrogen acceptor” and therefore, will not proceed, no matter how abundant the enzyme ADH in the reaction mixture is. It should be noted that once NAD has become NADH, the activity of ADH will slow down. The cell or the fluid will accumulate NADH. Fortunately, there are other reactions in the body that will use NADH. In these other reactions the NADH will be converted to NAD, which then can help enzymes like ADH to do their work to degrade other ethanol molecules.

To provide the condition where acetaldehyde is then the focus of elimination, the present technology will include in the preparation not only ADH but also aldehyde dehydrogenase (ALDH). Therefore, a crude extract of yeast extract will be preferable, without the need to “purify” the ADH or the ALDH from the crude extract, which may contain other useful enzymes as well. The preparation of the present technology can be supplemented with a high dose of NAD (and not NADH) or other co-factors (or precursors) which will reinforce the formation of NAD or NADP inside the cell, to drive the alcohol to acetaldehyde (which is harmful) and at the same time drive the acetaldehyde toward the direction of acetate which is harmless and easily removed by the body.

The co-factor NAD will be converted to NADH during the process of converting acetaldehyde to acetate, as indicated in the left side of FIG. 2. Due to abundance of the enzyme alcohol dehydrogenase ADH in the body, a lot of NAD will be converted to a lot of NADH. An abundance of NADH will help the “reverse reaction” i.e. the enzyme ADH will convert the acetaldehyde back to alcohol (the right side of FIG. 2). Therefore, the preparation of the present technology contemplates the inclusion of compounds that will use up the co-factor NADH. The phrase “using up” is meant to refer to the conversion of NADH back to NAD with appropriate enzyme reactions. One such useful reaction in the “reverse direction” will involve a combination (i.e. a mixture) of a high concentration of D-fructose and sorbitol dehydrogenase: this reaction consumes the fructose which will at the same time convert NADH to NAD.

It should be noted that a high concentration of NAD will favor both the activity of the alcohol dehydrogenase (ADH) as well as the next enzyme called Acetaldehyde Dehydrogenase (ALDH). Therefore, a high concentration of NAD is a “good” situation in the desire to remove both the ethanol and then the cancer-causing acetaldehyde (with the end-product being acetate). See FIG. 3 to view how both ADH and ALDH need NAD as co-factor to oxidize alcohol to acetate. The only “worry” is that one molecule of NAD will convert to one molecule of NADH and therefore, in the absence of a system to deplete NADH, or regenerate NAD, the NADH concentration will increase when alcohol is converted to acetaldehyde. Therefore, the NADH concentration must be kept low, by the diversion of NADH (to NAD), so that there is not enough of NADH to push the alcohol dehydrogenase to go in the “wrong” direction (i.e. not going from acetaldehyde back to alcohol).

One example of a “diversion package” is an enzyme that uses NADH (as co-factor) with a substrate that will also favor the direction of a reaction which deletes NADH. FIG. 4 illustrates how the reversible catalytic reaction of sorbitol dehydrogenase (SDH) can be used to mainly take the “reverse path” (which is the “favored path” in the present technology) to reduce the concentration of NADH.

It should be noted that most textbooks describe the activity of SDH as the activity driving D-sorbitol towards fructose (hence the name “sorbitol dehydrogenase). However, this enzyme is capable of the “reverse” reaction, as highlighted in FIG. 3. In the presence of a high concentration of D-Fructose and NADH, sorbitol will be formed which is highly soluble, harmless sugar which can be easily removed by the body. Again, the conventional description of the above reaction is that it is the “reverse” reaction, but in the present technology, this is the preferred reaction. To promote the conversion of fructose to sorbitol, the present technology can include a high concentration of fructose in the detox preparation, although any other compounds which promotes the consumption of NADH (to produce NAD) could be used.

It should be noted that sorbitol dehydrogenase (SDH) typically has very low activity in the plasma. Its main source is the hepatocyte. In alcoholics, it is probable that there is some degree of hepatic injury where SDH is released into the blood. Therefore, both the added compound (fructose) and the endogenous condition (presence of SDH in the cell or in the blood) are helpful in the regeneration of NAD from NADH. In other words: in addition to the fructose added to the detox preparation (which will be ingested orally to lower the concentration of NADH in the digestive system), the SDH in the liver or the blood will revert the NADH that is already in the blood or in the cell into NAD. Subsequently, the NAD drive both the ADH and the ALDH towards the elimination of both alcohol and acetaldehyde in the cell or in the blood (toward acetate, and not toward ethanol).

Although the present technology uses fructose and SDH as an illustration of a “diversion path” for NADH (so that less NADH is available for the conversion of acetaldehyde back to alcohol), there are other combinations of compounds and enzymes that will use up NADH, which will all help to drive the reaction of acetaldehyde toward acetate. It should be noted that sorbitol and fructose are “generally-known-as-safe” compounds and are therefore, acceptable to both oral intake as well as intravenous preparations.

The present description will mention the term “reinforced” and “double booster” or “booster” in this disclosure. The terms and the use of these compounds will be described as follows:
1. There is a limited concentration of NAD in the body. Therefore, after consumption of a large quantity of alcohol, the limiting factor is NAD (not the enzyme ADH) i.e. there are not enough NAD for the processing of the alcohol in the body even though the body has enough enzyme to do the job.
2. It has been found that two co-factors (Nicotinamide Riboside, NR and Nicotinamide Mononucleotide, NMN) both can increase the concentration of NAD in the body (in the fluid phases and inside cells). For an excellent review, see Mehmet et al “Nicotinamide Riboside—The Current State of Research and Therapeutic Uses” in Nutrients 202, June: 12(6): 1616.

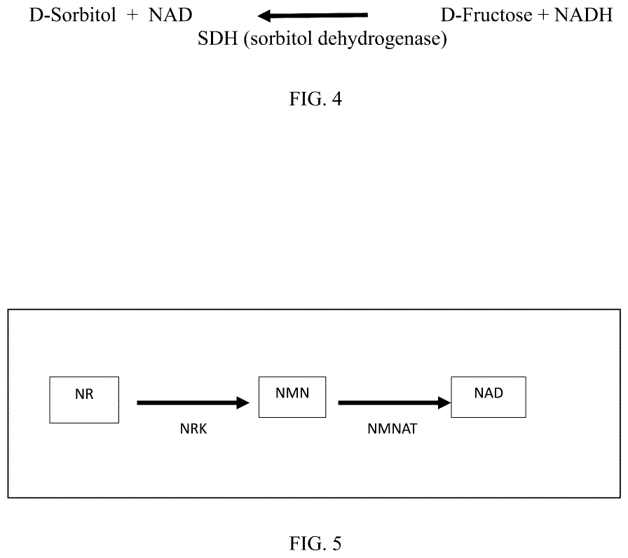

The relationship between NR, NMN and NAD can be simplified in the following and in reference with FIG. 5 that illustrates the buildup of NAD from NR or NMN.

NR can be converted to NMN by the enzyme Nicotinamide Ribose Kinase (NRK). NMN can be converted to NAD by the enzyme NMN Adenyltransferase (NMNAT).

The difference between NR and NMN is that NMN has a phosphate group that is not in the NR.

Both NR and NMN appear to be safe to human consumption and can be taken by mouth up to 1000 mg per dose per day with no ill effects.

The present description can call any molecule that can increase the concentration of NAD as “booster” including NR and NMN, because an increase in NR will increase the activity of NR kinase, leading to an increase in the concentration of NAD later. An increase in the concentration of NMN will immediately increase the concentration of NAD in the body.

There is a big debate among nutritionists whether the intake of NR is better than the intake of NMN, or whether it is more “economic for the body” if the patient takes NMN. The argument is the following: (a) NMN is one step close to the production of NAD, therefore, saving the body some energy to first convert NR to NMN. However, initially there are questions whether the body has enough of the enzyme NMN Adenyltransferase or whether the presence of the phosphate group in the NMN will hinder the uptake of this phosphorylated compound into the cells. (b) However, the body seems to be able to absorb the NMN in large quantities, even in the digestive system, without problem. Therefore, most nutritionists now favor the ingestion of NMN over the ingestion of NR.

In this disclosure the use of the term “reinforced” is to mean the following: that the concentration of one cofactor (e.g. NMN) is increased because of the action/presence/concentration of another cofactor (e.g. NR), which is the “reinforcer.” The present description will call NR as a “co-factor”, or “substrate” when talked about the conversion of NR into NMN. Similarly, the present description will call NMN as a “co-factor” or “substrate” when talked about the conversion of NMN into NAD. However, both NR and NMN will eventually become NAD, and therefore, the present description may call both NR and NMN “precursors” when referencing the conversion of NR or NMN into NAD.

Referring to FIG. 5, it can be seen that the co-factor NMN can be catalyzed into NAD or into NR. In the present technology, it is preferred that NMN is converted into NAD and not NR. However, as stated above, the enzyme reactions do not depend only on the presence of the correct enzymes, but mostly on the relative abundance of the “substrate” versus the “product.”

In situation A: if the enzyme NMN Adenyltransferase is in high concentration, NAD will build up quickly. A high NAD (the product initially) will favor the reverse direction, i.e. conversion of NAD back to NMN and may be even into NR.

In situation B: if the enzyme NR kinase is present in high concentrations, it may catalyze NR into NMN, but when NMN is built up, the enzyme will “reverse-catalyze” the NMN back to NR. Therefore, it would be wise to first build up the concentration of NR, so that the reactions will go mainly toward the right side of FIG. 5 (with NAD being the “final product”).

In this situation (situation C) a high concentration of NR will promote the formation of NMN. A buildup of NMN, however, will still favor the conformation of NAD (and not NR) because NR is still in a commandingly high concentration (therefore, all reactions will move from the left side to the right side of FIG. 5). Even when NAD is building up, the overall enzyme reaction direction will still favor the “conversion of NR” toward the buildup of NMN, which is then converted to NAD.

If one uses only NR the scheme will work, but a very high concentration of NR is needed. It is disclosed in the present technology to use both NR and NMN so that the following can occur: (1) NR will proceed to be converted to NMN because of the high concentration of NR; (2) a buildup of NMN will favor its conversion to NAD (and not NR) because the presence of a high concentration of NR will block the reverse direction (i.e. inhibit the movement from right side to the left side in FIG. 5). The present technology can propose to use twice the mass of NR in the detox preparation compared to the weight of NMN in the same preparation. However, this ratio can be adjusted to the need of the patient.

Summarizing the discussion above, the molecule NR is the “reinforcer” forcing the reaction to go toward the right side, from NR towards NMN, and at the same time from NMN toward NAD, which is the most desired molecules to eliminate alcohol in the body.

To use a sports analogy, the NR is acting like the “starting block” which will help a runner propel himself or herself forward in a race in the correct direction toward the desired finish line (to make more NAD).

In this disclosure, NAD is used interchangeably with NADP because many enzymes (though not all) can use either one of these two co-factors.

The reaction of NAD being converted to NADH is a “reductive” or “reduction” reaction (as opposed to “oxidative” reaction) because a hydrogen atom has been added to the NAD. The “reverse reaction” from NADH to NAD is an oxidative reaction for the co-factor NADH because a hydrogen atom has been removed from NADH. Since the word “reduction” is often used (in ordinary discussions) to mean “decrease in concentration” (or in number, or in quantity) and not related to the biochemistry of oxidative/reductive reactions, in this disclosure (to avoid misunderstanding) the present description may call the step of NADH being converted to NAD (the useful form in the elimination of alcohol from the body) as a “co-factor reverse reaction” (which is good for the purpose of the present technology). One example has been depicted in FIGS. 3 and 4 where the enzyme sorbitol dehydrogenase can catalyze a high concentration of NADH or fructose, or both (in the reverse sense to the name of the enzyme “sorbitol dehydrogenase”) into NAD and sorbitol.

The term “reverse-build-up” of NAD will mean using the same enzyme (e.g. sorbitol dehydrogenase) to conduct the reaction, which is in the reverse direction than usual, for the purpose of the “build-up” of the concentration of NAD (which is done by the lowering of the concentration of NADH which is in this case a substrate for the same enzyme, the reaction being conducted in the “reverse” direction). Both fructose and sorbitol are very safe sugars that can be consumed orally, or used intravenously with no apparent harm by normal healthy persons. The co-factor NAD (or NADP) is present in limited concentrations in the body. In any enzyme reaction, e.g. the catalysis of ethanol and NAD into acetaldehyde and NADH, the limiting substrate is typically NAD (and not the concentration of alcohol). Therefore, increasing the concentration of NAD in the body or in any cell will increase the rate of the reaction to eliminate alcohol.

However, NAD is not as stable as its precursors (NR or NMN) and may be taken into the cells as easily as NR or NMN. Therefore, the present technology can intend to use one or more precursors to NAD (and NADP) which can be consumed by oral intake, which can be readily converted into NAD or NADP in the cells. The precursors can be NR (nicotinamide riboside) or NMN (nicotinamide mononucleotide). Either one or both can be taken orally in large amounts (up to or more than 1000 mg per day over many years) without any detectable undesirable side effects. It was noted that people who took these precursors daily would regain their youthfulness or energies, because their cells have now been replenished with NAD or NADP which is essential for their daily metabolic needs. However, no prior art appears to teach what this disclosure teaches, i.e. that the intake of high concentrations of NR or NMN, or both, in the presence of either ADH, or ALDH, or both, can greatly benefit the person who is intoxicated with alcohol.

Although exact dimensions or concentrations of ingredients and other features may be disclosed in the present technology, it will be readily apparent that people skilled in the art can use other dimensions or concentrations or variations to achieve similar results, which are but variations of the present technology and in violation of the body and spirit of this disclosure.

1. It has been discovered according to the present technology that a detoxification preparation for ingested alcohol, comprising a mixture of enzymes that breaks down alcohol and alcohol-byproducts, and co-factors (or co-enzymes) for these reactions can be prepared in a liquid form to be consumed orally by a person in need of removing the toxic effects of alcohol consumption, in an amount sufficient to result in detoxification from alcohol consumption.

2. It has been discovered according to the present technology that a detoxification preparation for ingested alcohol, comprising a mixture of enzymes that breaks down alcohol and alcohol-byproducts, and co-factors (or co-enzymes) for these reactions, can be prepared in a semi-liquid form such as a soft or hard candy, to be consumed orally by a person in need of removing the toxic effects of alcohol consumption, in an amount sufficient to result in detoxification from alcohol consumption.

3. It has been discovered according to the present technology that a detoxification preparation for ingested alcohol, comprising of a mixture of enzymes that breaks down alcohol and alcohol-byproducts, and co-factors (or co-enzymes) for these reactions, can be prepared in a powder or solid form to be stored in a moisture-resistant pouch which can be opened so that the said mixture can be dissolved in a liquid and then consumed orally by a person in need of removing the toxic effects of alcohol consumption, in an amount sufficient to result in detoxification from alcohol consumption.

4. It has been discovered according to the present technology that a detoxification preparation for ingested alcohol, comprising of a mixture of enzymes that breaks down alcohol and alcohol-byproducts, and co-factors (or co-enzymes) for these reactions, can be prepared in a powder or solid form to be stored in a “gel capsule” which can be swallowed by a person in need of removing the toxic effects of alcohol consumption, in an amount sufficient to result in detoxification from alcohol consumption.

5. It has been discovered according to the present technology that a detoxification preparation can be prepared, comprising of a mixture of enzymes and co-factors (or co-enzymes) as mentioned in the various physical forms above, comprising at least one enzyme that will lower the concentration of alcohol in the body and another enzyme that will lower the concentration of acetaldehyde in the body, and at least one co-factor such as NAD which will accelerate the action of one or both of the said enzymes in the direction away from the formation of alcohol and in the direction of the formation of acetate, so that the concentration of alcohol and acetaldehyde will be lowered in the body of the person who consumes the detoxification preparation.

6. It has been discovered according to the present technology that a detoxification preparation can be prepared, comprising of a mixture of enzymes and precursors to the co-factors (or co-enzymes) as mentioned in the various physical forms above, comprising at least one enzyme that will lower the concentration of alcohol in the body and another enzyme that will lower the concentration of acetaldehyde in the body, and at least one precursor to the co-factor which is either NAD or NADP which will accelerate the action of one or both of the said enzymes in the direction away from the formation of alcohol and in the direction of the formation of acetate, so that the concentration of alcohol and acetaldehyde will be lowered in the body of the person by the increase in NAD or NADP in the body of the person who consumes the detoxification preparation.

7. It has been discovered according to the present technology that a detoxification preparation can be prepared, comprising of a mixture of enzymes and precursors to the co-factors (or co-enzymes) as mentioned in the various physical forms above, comprising at least one enzyme that will lower the concentration of alcohol in the body and another enzyme that will lower the concentration of acetaldehyde in the body, and at least one precursor such as NR (nicotinamide riboside) or NMN (nicotinamide mononucleotide) to the co-factor which is either NAD or NADP which will accelerate the action of one or both of the said enzymes in the direction away from the formation of alcohol and in the direction of the formation of acetate, so that the concentration of alcohol and acetaldehyde will be lowered in the body of the person by the increase in NAD or NADP in the body of the person who consumes the detoxification preparation.

8. It has been discovered according to the present technology that a detoxification preparation can be prepared, comprising of a mixture of enzymes and precursors to the co-factors (or co-enzymes) as mentioned in the various physical forms above, comprising at least one enzyme that will lower the concentration of alcohol in the body and another enzyme that will lower the concentration of acetaldehyde in the body (these two enzymes being called “detox” enzymes), and at least two precursors such as NR (nicotinamide riboside) and NMN (nicotinamide mononucleotide) to the co-factor which is either NAD or NADP, such that the combined effect of these two precursors will result in a high concentration of NAD or NADP in the cells capable of conducting the activities of the detox enzymes in the direction away from the formation of alcohol and in the direction of the formation of acetate, so that the concentration of alcohol and acetaldehyde will be lowered in the body of the person by the increase in NAD or NADP in the body of the person who consumes the detoxification preparation.

9. It has been discovered according to the present technology that a detoxification preparation can be prepared, comprising of a mixture of enzymes, precursors of the co-factors (or co-enzymes, as mentioned above in various physical forms), and substrates helpful to the reverse-build-up of the useful co-factors (such as NAD), comprising (a) at least one enzyme that will lower the concentration of alcohol, (b) at least one enzyme that will lower the concentration of acetaldehyde, (c) at least one precursors (such as NR, or NMN, or both) to the co-factor such as nicotinamide-adenine-dinucleotide (NAD) that will accelerate the action of the enzymes in the direction of acetate, and away from the formation of alcohol, (d) at least one substrate (e.g. fructose) that will increase the formation of NAD from NADH, so that the overall result is that the concentration of alcohol and acetaldehyde will be lowered in the body of the person who consumes the detoxification preparation, due to the availability of a high concentration of NAD in the body.

### EXPERIMENTS

### Experiment 1: Effect of a High Concentration of NAD or NADP on the Effective Rate of Enzyme Activity of Alcohol Dehydrogenase

### Introduction:

All enzyme activities are studied under a “standard condition” where the concentrations of the substrates are “within normal limits” (i.e. physiological condition) while the concentration of the enzyme is the “variable”, being added to the reaction mixture at various concentrations. If the purpose of the study is to find out the “activity” of the enzyme preparation (e.g. how many units of enzymes per mg of protein in the crude extract) a portion of the crude extract is added to the reaction mixture and the rate of formation of the product (usually a colored substance) is measured. At the same time, known concentrations of purified enzymes is added to the same reaction mixture to generate a “standard curve”.

A comparison of the curve generated by the enzyme in the crude extract with the curve generated by the pure enzyme preparation will allow calculation of the “number of units of the enzyme per mg protein” in the crude extract (the concentration of protein in the crude extract will be measured by a separate experiment).

It should be noted that the entity of interest should always be the material in “limited amounts”. For example, if alcohol dehydrogenase is to be measured, the alcohol and NAD would be “more than enough” (non-limiting) for the reaction to go on (for the duration of the experiment, typically 10 to 30 minutes): thus a 2× concentration of ADH in one preparation will generate a 2× steeper curve (the gradient of the slope) than another preparation that has 1× concentration of ADH.

Another example will be a study on the concentration of alcohol in a solution. In that case, the concentration of NAD and that of ADH must be non-limiting; so that a solution with 5% alcohol will generate a curve half as rapid as another solution with 10% alcohol.

Due to the fact that NAD is often regarded as “the co-factor” of most enzyme reaction studies, it is almost never used as a rate-limiting factor, because it has to be the non-limiting partner so that the other “more important” components of the reaction can be measured (e.g. alcohol, or ADH).

In a commercial product, cost is always an important consideration. The enzyme ADH is expected to be the most costly item in the detox preparations as discussed in the present technology. A can of beer of 12 oz (360 mL) (5% alcohol) contains 18 gram of “pure alcohol.” The molecular weight of ethanol is 46 dalton. Therefore, there are 0.4 moles of alcohol in one can of beer.

To eliminate all the ethanol molecules in that beer within 10 minutes would require a high concentration of ADH according to the conventional wisdom.

The activity of 1 unit of ADH is defined as the amount of ADH capable of removing 1 micromole of ethanol per minute (under standard conditions of pH 8.8 at 25° C.). Therefore and in the exemplary, the number of units to completely remove 400 millimole of ethanol in 10 min is shown in Equation 1

Number of units=400/(0.001×10) Equation 1

which is 40,000 units of ADH under standard conditions. Where 400 represents the amount of ethanol in millimoles, 0.001 is in millimoles and represents 1 micromole of ethanol, and 10 represents the time to eliminate the ethanol molecules in minutes. Equation 1 generals calculates the number of units of the enzyme or co-enzyme needed to remove an amount of ethanol in millimoles within a specific time in second, wherein a millimole value of ethanol is divided by a time value in minutes.
The cost of ADH for 40K units of ADH (e.g. from Sigma-Aldrich, A7011) will be more than $60.00 which will set the price of the detox preparation in this disclosure to be above what most consumers will pay. Even if one does not aim at removing 100% of the ethanol in a can of beer, but only 10%, the cost for ADH alone will be $6.00 again far above what the average alcoholic drinker would want to pay.

Therefore, the aim of the experiment below is to evaluate if the “apparent” activity of the ADH can be increased, not by increasing the concentration of the enzyme, but by increasing the concentration of the co-factor NAD or NADP, so that the cost of the detox preparation can be affordable to the average consumer. The cost of NAD or its precursors are much lower than the cost of the enzyme.

### Purpose:

To study if a higher concentration of NADP results in higher (observable) ADH activity. The concentration of ADH being kept constant at 84 units per mL in the reaction mixture.

### Materials and Methods:

ADH (150 KU bottle, 300 U/mg) and NADP (100 mg bottle) were purchased from Sigma. 100% ethanol (EtOH) was purchased from VWR. Buffer used was Triz Base at 0.2 M, pH 8.2. The following solutions were made fresh and used within 1 hour:
NADP, 50 mM, in buffer
EtOH, 75% in buffer
ADH, 2 mg/mL in buffer

In the reaction mix, there are 2 final concentrations of NADP, i.e. 8.5 mM or 28 mM NADP. The concentration of ethanol is 2.5% (v/v). And 0.28 mg/mL ADH, equivalent to 84 U/mL.

TABLE 1

Sample Name

| | NADP 1x | NADP 3x | |
|---|---|---|---|
| Reagent | (mL) | (mL) | Blank (mL) |
| Buffer | 0.45 | 0.17 | 0.55 |
| NADP, 50 mM in | 0.12 | 0.4 | 0.12 |
| buffer | | | |
| EtOH, 75% in buffer | 0.024 | 0.024 | 0.024 |
| ADH, 2 mg/mL in | 0.1 | 0.1 | 0.1 |
| buffer | | | |
| Total | 0.7 | 0.7 | 0.7 |

Multiple samples were prepared as per Table 1. The assay was performed as follows:
1. Add buffer, NADP, and EtOH in the appropriate amounts to the quartz cuvette.
2. Place a quartz cuvette into the spectrophotometer.
3. At time minus 10 seconds, add 0.1 mL enzyme (2 mg/mL ADH or buffer blank) to the cuvette.
4. Mix by pipetting. Read OD340 at time zero.
5. Read OD340 every minute for 5 minutes. 6. Repeat for remaining samples.

### Results and Discussion:

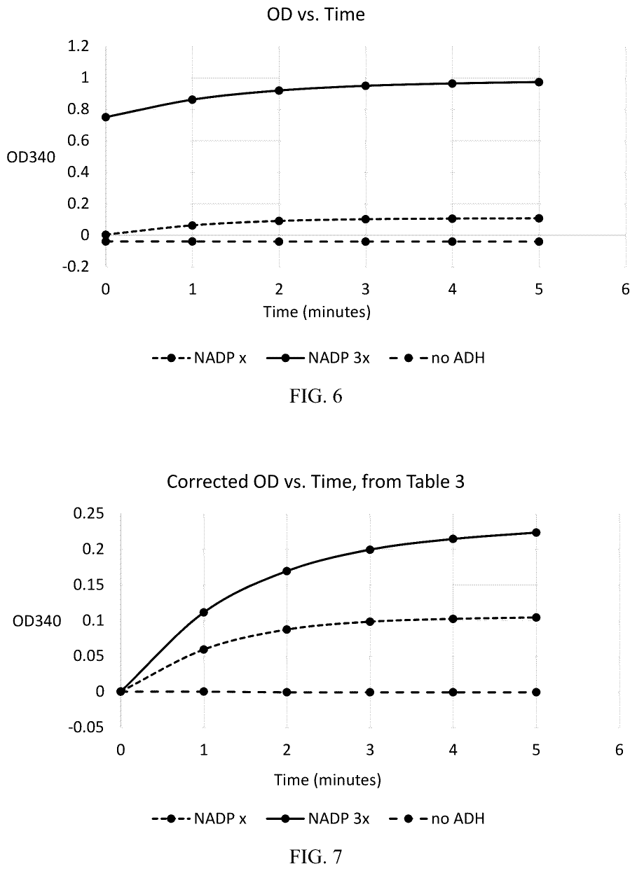

OD versus (vs.) time for the samples—NADP 1× and NADP 3× with final concentrations at 8.5 mM and 28 mM, respectively—is presented in Table 2 and as shown in FIG. 6 graphically illustrating OD of samples after subtracting OD at time zero vs. Time. OD at time zero is high for the NADP 3× sample, around 0.8 due to material in the cuvette that produces absorbance at OD340. OD vs. time is presented and plotted after subtracting OD at time zero in Table 3 and as shown in FIG. 7.

It can be appreciated that OD is an absorbance reading, and OD340 is an absorbance reading taken at 340 nm. Additional nanometer readings can be taken to verify the absorbance of ADH and/or ALDH.

These results show that a higher NADP concentration does have higher ODs at every time point, starting at 1 minute, when compared with the lower NADP concentration. In future studies, this trend will be further studied with an even higher concentration of NADP, and/or by using similar NAPD concentrations and reducing ADH concentrations.

TABLE 2
OD of samples vs. Time

| Tube | 0 min | 1 min | 2 min | 3 min | 4 min | 5 min |
|---|---|---|---|---|---|---|
| NADP 1x | 0.003 | 0.062 | 0.090 | 0.101 | 0.105 | 0.107 |
| NADP 3x | 0.749 | 0.860 | 0.918 | 0.948 | 0.963 | 0.972 |
| no ADH | −0.040 | −0.040 | −0.041 | −0.041 | −0.041 | −0.041 |

TABLE 3
OD of samples after subtracting OD at time zero vs. Time

| Tube | 0 min | 1 min | 2 min | 3 min | 4 min | 5 min |
|---|---|---|---|---|---|---|
| NADP 1x | 0 | 0.059 | 0.087 | 0.098 | 0.102 | 0.104 |
| NADP 3x | 0 | 0.111 | 0.169 | 0.199 | 0.214 | 0.223 |
| no ADH | 0 | 0 | −0.001 | −0.001 | −0.001 | −0.001 |

### Comments:

By using a 3.3× concentration of NADP (28 mM vs 8.4 mM) in the reaction mixture, the same conc of ADH is able to remove ethanol at about 2× the velocity in the higher NADP mixture when compared to the lower NADP mixture. This Experiment 1 is later repeated with 28 mM and 8.4 mM NADP but with lower concentrations of ADH, i.e. 28 units per mL (0.3×) and 8.4 units per mL (0.1×)—to increase the ratio of NADP to the quantity of ADH. It was found that even at 8.4 units of ADH per mL, a higher concentration of NADP will have a faster velocity (at least 2×) than that of the rate shown with 8.4 mM of NADP. In other words, the enzyme is able to function in much faster rates than is described under “standard condition” in vitro—suggesting that the limiting factor in vivo is the availability of NAD or NADP (versus the buildup of NADH and NADPH inside the cell) and not the ability of the enzyme ADH to break down the ethanol molecules.

Regarding the effect on increasing the NAD concentration inside cells after ingestion of a large amount of the stable precursor NR, which can be consumed orally and without bad taste: The molecular weight of NR is 255 daltons. Ingestion of 1000 mg of the precursor NR will mean a rise in the cellular level of NAD by 1/255 moles, or 4 millimoles. The volume of distribution is in a person who has ingested a large quantity of alcohol is not clear. However, if the NR is absorbed mainly in the GI tract, then the volume of distribution within these cells may be small: just for the sake of discussion, if the volume of fluids inside the cells along the gut is only 1 liter, then the “concentration” of this mass (4 millimole) of NAD in the small volume of cellular fluid will be 4 millimolar (i.e. 4 mM), which is a very high intracellular concentration. In contrast, the concentration of NAD inside the cell is only about 0.2 mM to 0.5 mM (Canto et al), which varies only 2-fold even under stressful conditions.

The concentration of NAD inside the cell has been studied. In an article by Canto et al published in Cell Metab. 2015 Jul. 7; 22(1): 31-53, titled “NAD+ metabolism and the control of energy homeostasis—a balancing act between mitochondria and the nucleus,” the authors stated (section 2.3 “Cell compartmentalization of NAD+”) the following: In general, intracellular NAD+ levels are maintained between 0.2 and 0.5 mM, depending on the cell type or tissue. However, NAD+ levels can change, up to ˜2-fold, in response to diverse physiological stimuli.

Regarding the ratio of NADP vs NADPH, their relative concentrations in the cell have also been studied. In an article published in Anal Biochem. 2019 May 1; 572: 1-8, titled “Assessment of NAD+ metabolism in human cell cultures, erythrocytes, cerebrospinal fluid and primate skeletal muscle” by Demarest et al, the authors stated that “The NADP/NADPH ratio was ˜1:1 for both HEK-293T cells and RBCs.” This strongly suggests that a slight disturbance in the ratio in favor of the concentration of NADP (or NAD) will greatly facilitate the enzymatic reaction in favor of the elimination of ethanol inside the cell. Conversely, a relative decrease in the concentration of NADH or NADPH (relative to the concentration of NAD, or NADP) will also move the reaction towards the desirable direction: i.e. the elimination of ethanol toward the formation of acetate.

The effect of high NADP was studied on the reaction rate of the enzyme ADH. However, the enzyme reaction of ALDH is essentially the same, using NAD as the hydrogen-receiver (to become NADH). It is therefore expected that a high concentration of the precursors NR or NMN would have a great benefit on the conversion of acetaldehyde to acetate.

In the exemplary, there can be another situation of a “reinforced booster” except that the reinforcement applies to the enzymes as well as to the substrates. Referring to FIG. 1, it should be noted that since ADH and ALDH are two enzymes working in sequence, any increase in the activity of ALDH (under the influence of a high concentration of NAD—a condition generated by the present technology) will not only increase the rate of formation of acetate and at the time the decrease in the concentration of acetaldehyde; the lowering of the concentration of acetaldehyde will definitely facilitate the reaction rate of the first enzyme ADH in degrading ethanol into acetaldehyde. Therefore, using the concept of “reinforced” enzyme reactions, as in the discussion above regarding NR and NMN, the high activity of one enzyme will lead to the improved activity of another enzyme, both working in the same direction of removing ethanol and producing acetate. In terms of the “substrates” the same thing happens under the condition generated by the present technology: the presence of a high concentration of ethanol (in the presence of a high concentration NAD, a condition created by the present technology) will increase the rate of formation of the “middle product” or “intermediate product” which is acetaldehyde. Normally (without the benefit of the present technology), with a high concentration of ethanol that needs to be degraded, NAD is consumed quickly and acetaldehyde is built-up. However, in the present technology, the concentration of NAD is no longer the limiting factor and the concentration of acetaldehyde is kept low. All these conditions favor the conversing of ethanol to acetate. It may also be said that ethanol is the “starting block” (in an analogy of a competing runner in a race) which will help the reactions to go only to the right side, which is the formation of acetate (the “finish line” of a race).

To recap: In the detox preparation of the present technology, both ADH and ALDH can be utilized, and the intracellular concentration of NAD can be supplemented by providing a high concentration of NR or NMN in detox preparation, so that NAD is not the limiting factor. This condition favors both the coordination of the enzymes and the reinforcement of the substrates to remove ethanol molecules and to the formation of acetate molecules. With a high intracellular NAD concentration (created by the ingestion of its precursors in the present technology) the degradation of acetaldehyde into acetate can proceed without impediment, but indeed be accelerated. At the same time, the relatively low concentration of acetaldehyde will “stimulate” the reactivity of ADH in degrading ethanol even faster than normal (into acetaldehyde). Therefore, both enzyme reactions are favored to move toward the “right side” which is the formation of acetate under the conditions generated by the high concentration of NAD as disclosed here.

Therefore, in the concept of a reinforced booster, one may consider the ethanol (high concentration) to be the “starting block” (substrate) to help the runner (ADH) to move only in the desired direction (toward the right side or the formation of acetate, FIG. 1) while at the same time “encouraging” the next enzyme ALDH to catalyze the reaction also to the right side and not to the left side (resulting in a substrate back to ethanol).

One may also say that the high NAD concentration in the cell, created by the present technology is both (a) the starting block substrate (because it is truly a substrate in the ethanol-conversion-to-acetaldehyde step) and that it is also (b) the “baton-receiver” (in a baton-relay race) because its high concentration in the cell helps the conversion of acetaldehyde molecules (in the middle of the race) into acetate molecules (the finish line of the race).

It can thus be concluded that these experimental data of the present technology suggests that there is no need to include a super-high concentration of the enzyme ADH or ALDH in this detox preparation, provided a high concentration of NAD or NADP can be produced inside the cell by the intake of a high concentration of NP and NMN provided by the detox preparation of the present technology. The appropriate amount of both enzymes in the detox preparation of the present technology will augment the function and effectiveness of the endogenous concentration of ADH and ALDH already existing in the digestive system (the gut and the liver) of the alcohol drinker. The benefit of the present technology detox preparation will be easy to observe since the activity of the drinker's own enzymes and cofactors are often insufficient to overcome the intoxicating effect of the amount of alcohol consumed. In particular, the middle (intermediate) product acetaldehyde is known to be highly toxic and may be a cancer-producing agent. Rapid removal of acetaldehyde by the use of the present technology detox preparations will be of great importance to not only the individual drinker, but to society overall in lowering the cost of cancer incidence and the cost of treatment.

### Experiment 2: Preparation of Counter-Alcohol Enzyme/Co-Factor/Precursor Mixture in a Pouch or a Gel Capsule

### Introduction:

Although ADH and ALDH can be obtained from a number of species other than yeast, it can be preferable to obtain them from crude extracts of yeast, which can be ingested without problem as baking products (bread etc.). The crude extracts can be lyophilized into dry powder. The present technology can use fructose or trehalose as the excipient for the lyophilization process.

### Materials and Methods:

The present technology lyophilized all the liquid preparations of the enzyme solutions into dried powder. Then they are mixed by weight with the rest of the ingredients which are already in powder form and package them into (a) pouches or (b) gel capsules. The present technology may include other ingredients, such as fruity favoring products (to promote a good after-taste for drinkers who often crave a certain sweet product after consuming alcohol). The list is provided below in Table 4.

### Results:

The dry powder containing all the desired enzymes and co-factors can be easily dissolved in water or any drinks before consumption by the person in need of detoxification resulting in a quick resolution from the effects of alcohol consumption. Or the detox preparations can be opened or consumed after the alcoholic consumption. During experimentation, a volunteer was asked to take 2 capsules after 3 beers and this first volunteer reports good results of less intoxication. Another volunteer was asked to open a package of the detox package and dissolve it in water and drink it after 3 beers. The second volunteer also reports good detox effects from the present technology product.

### CONCLUSIONS

Storage of the detoxification preparation of counter-alcohol mixtures in a solid powder-form offers many advantages. It is light weight and can be kept in room temperature for a long time in a sealed pouch or other standard storage bags. Upon opening, the content can be dissolved in water or any other drinks to be consumed without trouble. The dissolved mixture works immediately just as good as similar preparations manufactured in ready-to-use liquid form or in the semi-liquid form as candies. It is expected that the gel-capsule format to be most popular because of the convenience to take capsules after an event of heavy drinking.

TABLE 4
Examples of the contents in gel capsules
and in moisture-resistant pouches.

| | WEIGHT PER | WEIGHT PER |
|---|---|---|
| CONTENT | CAPSULE, mg | PACKAGE, mg |
| Yeast Crude Extract | 10 | 50 |
| Estimated Alcohol Dehydrogenase in | 100 units | 500 units |
| yeast crude extract (ADH) | | |
| Estimated Acetaldehyde | 30 units | 150 units |
| Dehydrogenase in yeast crude extract | | |
| (ALDH) | | |
| Nicotinamide Riboside (NR) | 300 | 1500 |
| Nicotinamide Mononucleotide | 100 | 500 |
| (NMN) | | |
| Fructose | 200 | 1000 |

While Table 4 provides examples of the weight of various components per capsule or per package, the exact weights of the various components or additional components in the commercial products may vary depending on the economy of production and the preferences of the buyers. In particular, the weight of crude yeast extracts per capsule may vary between 1 mg to 100 mg; the estimated ADH content may vary from 10 unit to 1000 units; the estimated units of ALDH may vary from 3 to 300 units; that of NR, NMN and Fructose may vary from 30 to 3000 units, 10 to 1000 units, and 20 to 2000 mg, respectively. The content per package may also vary from 10% that of each content per package listed in Table 4 to 1000% of the weight or units listed in Table 4.

While embodiments of the detoxification preparations with reinforced boosters to treat alcohol intoxication have been described in detail, it should be apparent that modifications and variations thereto are possible, all of which fall within the true spirit and scope of the present technology. With respect to the above description then, it is to be realized that the optimum relationships for the elements of the present technology, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present technology.

Therefore, the foregoing is considered as illustrative only of the principles of the present technology. Further, since numerous modifications and changes will readily occur to those skilled in the art, it is not desired to limit the present technology to the exact construction and operation shown and described, and accordingly, all suitable modifications and equivalents may be resorted to, falling within the scope of the present technology.

What is claimed as being new and desired to be protected by Letters Patent of the United States is as follows:

1. A composition for converting alcohol to acetaldehyde and/or acetate, the composition comprising:
a therapeutically effective amount of one or more first enzymes; and
a therapeutically effective amount of one or more second enzymes different to that of the first enzymes.

2. The composition according to claim 1, wherein the first enzymes is selected from the group consisting one of or any combination of alcohol dehydrogenase (ADH), a derivative of alcohol dehydrogenase, and an analog of alcohol dehydrogenase.

3. The composition according to claim 2, wherein the alcohol dehydrogenase is alcohol dehydrogenase in yeast crude extract.

4. The composition according to claim 1, wherein the second enzymes is selected from the group consisting one of or any combination of aldehyde dehydrogenase (ALDH), a derivative of aldehyde dehydrogenase, an analog of aldehyde dehydrogenase, mitochondrial aldehyde dehydrogenase (ALDH2), a derivative of mitochondrial aldehyde dehydrogenase, and an analog of mitochondrial aldehyde dehydrogenase.

5. The composition according to claim 4, wherein the aldehyde dehydrogenase or the mitochondrial aldehyde dehydrogenase is aldehyde dehydrogenase in yeast crude extract.

6. The composition according to claim 1 further comprising a therapeutically effective amount of a co-factor configured to increase a concentration of any one of or any combination of Nicotinamide Adenine Dinucleotide (NAD) or Nicotinamide Adenine Dinucleotide Phosphate (NADP) in vivo.

7. The composition according claim 6, wherein the co-factor is selected from the group consisting one of or any combination of Nicotinamide Riboside (NR), and Nicotinamide Mononucleotide (NMN).

8. The composition according to claim 6, wherein a concentration of the co-factor in the composition is configured to prevent conversion of acetaldehyde to alcohol or acetate to acetaldehyde.

9. The composition according to claim 1 further comprising an effective amount of any one of or any combination of a sugar and a third enzyme different to that of the first and second enzymes.

10. The composition according to claim 9, wherein the sugar is fructose, and the third enzyme is sorbitol dehydrogenase (SDH).

11. The composition according to claim 1, the composition is selected from the group consisting of a pharmaceutical composition, a dietary supplement, a nutraceutical, a medicament, a liquid composition, a confection, and a powder.

12. The composition according to claim 1 further comprises a pharmaceutically acceptable excipient.

13. The composition according to claim 1, wherein an amount of the composition is a prophylactically effective amount.

14. The composition according to claim 1, wherein a number of units of any one of or any combination of the first enzymes and the second enzymes is based on a value associated with a curve generated by the first and second enzymes, respectively, in a crude extract with a value associated with a curve generated by a pure enzyme preparation.

15. The composition according to claim 14, wherein the number of units is based on a millimole value of ethanol divided by a time value in minutes.

16. A method for detoxify an effect of alcohol consumption in a subject, the method comprising consuming the composition of claim 1 by the subject before and/or after consuming ethanol.

17. The method according to claim 16, wherein:
the first enzymes is selected from the group consisting one of or any combination of alcohol dehydrogenase (ADH), a derivative of alcohol dehydrogenase, an analog of alcohol dehydrogenase, and alcohol dehydrogenase in yeast crude extract;
the second enzymes is selected from the group consisting one of or any combination of aldehyde dehydrogenase (ALDH), a derivative of aldehyde dehydrogenase, an analog of aldehyde dehydrogenase, mitochondrial aldehyde dehydrogenase (ALDH2), a derivative of mitochondrial aldehyde dehydrogenase, an analog of mitochondrial aldehyde dehydrogenase, and aldehyde dehydrogenase in yeast crude extract.

18. The method according to claim 16 further comprising an effective amount of a co-factor configured to increase a concentration of any one of or any combination of Nicotinamide Adenine Dinucleotide (NAD) or Nicotinamide Adenine Dinucleotide Phosphate (NADP) in vivo, wherein a concentration of the co-factor in the composition is configured to prevent conversion of acetaldehyde to alcohol or acetate to acetaldehyde, and wherein the co-factor is selected from the group consisting one of or any combination of Nicotinamide Riboside (NR), and Nicotinamide Mononucleotide (NMN).

19. The method according to claim 16 further comprising an effective amount of any one of or any combination of fructose, and sorbitol dehydrogenase (SDH).

20. A method for in vivo reducing acetaldehyde levels and/or increasing a rate of catabolism of acetaldehyde, the method comprising, contacting at least one cell with an effective amount of the composition of claim 1 that modulates an enzymatic activity of an acetaldehyde dehydrogenase found in the at least one cell.
